(19) United States
(12) Patent Application Publication
(10) Pub. No.: US 2006/0112958 A1
(43) Pub. Date: Jun. 1, 2006

(54) Portable vapor inhaler

(76) Inventors: Jeffrey J. Fisher, Ada, MI (US); Carol J Watzke, Hopkins, MN (US); Miton W Anderson, Eagan, MN (US); Gustav R Fenton, Minneapolis, MN (US); Todd W Schansberg, Jordan, MN (US)

Correspondence Address: Fulbright & Jaworski, 2100 Ids Center, 80 South Eighth Street, Minneapolis, MN, 55402-2112, US

(21) Appl. No.: 10/537,854

(22) PCT Filed: Dec. 5, 2003
(86) PCT No.: PCT/US03/38661

§ 371 Date: Jun. 7, 2005

Related U.S. Application Data
(60) Provisional application No. 60/432,119, filed on Dec. 9, 2002.

(51) Int. Cl. A61M 16/10; A61M 15/00

(52) U.S. Cl. 128/203.16; 128/203.17

(57) ABSTRACT

The present invention is a portable vapor inhaler (10) for improving the breathing of those suffering from nasal congestion. The invention includes a vapor-concentrating lid (12) and an effervescent composition (14). In further embodiments the invention may include a reservoir. Hot water is placed in the reservoir along with the effervescent composition and the vapor-concentrating lid is placed thereon. The hot water creates a humid vapor in the reservoir and beneath the vapor-concentrating lid. The positive pressure created by the effervescent composition then forces the humid air out through the vapor-concentrating lid and is then inhaled by the user.

### PRIORITY

The present invention claims priority from prior U.S. Provisional Patent Application Ser. No. 60/432,119, filed on Dec. 9, 2002.

### TECHNICAL FIELD

This invention relates to an application for decongesting nasal passages. More particularly, this invention relates to a portable vapor inhaler that helps to improve breathing by decongesting nasal passages.

### BACKGROUND

Nasal congestion is a significant problem and a characteristic symptom of various conditions, including allergies and colds. Nasal congestion interferes with breathing and makes the sufferer of congestion uncomfortable. Relieving nasal congestion can help the sufferer of a cold or allergy feel better, allow them to breathe more easily, and to function better during work or recreation.

One way to treat nasal congestion can be with pharmaceutical materials. Treating nasal congestion with pharmaceutical materials, however, may have deleterious effects. Pharmacological side effects may leave a person feeling listless, sleepy, excited or inattentive.

One traditional non-pharmaceutical method of relieving nasal congestion is humid vapor therapy. Humid vapor therapy can be provided by a steam vaporizer. A steam vaporizer provides highly moistened air that can be inhaled to drain accumulated mucus from nasal passages and sooth swollen, irritated nasal tissues. Steam vaporizers often add decongesting fragrance materials such as menthol or camphor to the humidified air to increase the effectiveness of inhaling this treated air.

One significant problem with steam vaporizers, or similar devices, is the fact that they are not portable. Hindrances to portability include size, weight and electrical requirements. Attempts to provide for similar benefits in portable form have taken two forms. In one approach, the decongesting fragrances are simply placed in a tube or medium and inhaled. This approach lacks the benefits of humidified air, which is especially beneficial during cold and allergy seasons when the ambient air tends to naturally lack humidity. The second approach is to devise miniaturized equipment that forces ambient air through or over water solutions, perhaps warmed by resistance heating or ultrasonics. These approaches have also proved unreliable.

### DISCLOSURE OF THE INVENTION

The present invention is a portable vapor inhaler for improving the breathing of those suffering from nasal congestion. One embodiment of the present portable vapor inhaler invention includes an effervescent composition and a vapor-concentrating lid for attachment to a standard sized cup.

A second embodiment includes a water reservoir, an attachable vapor-concentrating lid, and an effervescent composition, which, when added to the water, provides a positive internal pressure in the reservoir, forcing humidified air from the vapor-concentrating lid.

In another embodiment the portable vapor inhaler includes a vapor-concentrating lid and water reservoir as one complete unit.

In yet another embodiment, the effervescent composition may further contain decongesting fragrance materials to enhance the decongesting effect.

The present invention further may include a portable vapor inhaler comprising a reservoir, a vapor-concentrating lid that is removably attachable to the reservoir, and an effervescent composition.

In yet another embodiment, the system for the inhalation of humidified vapor comprises a reservoir, a vapor-concentrating lid that is removably attachable to the reservoir, and an effervescent composition.

The present invention also includes a method for the inhalation of humidified vapor comprising filling a reservoir with hot water, adding an effervescent composition to the hot water, the effervescent composition causing to be released an amount of gas such that a positive vapor pressure is created in the reservoir, connecting a vapor-concentrating lid to the reservoir, and breathing an amount of humidified air.

The present invention also includes a method for treating cold symptoms comprising filling a reservoir with hot water, adding an effervescent composition to the hot water, the effervescent composition causing to be released an amount of gas such that a positive vapor pressure is created in the reservoir, connecting a vapor-concentrating lid to the reservoir, and breathing an amount of humidified air.

The present invention also includes a method for treating allergy symptoms comprising filling a reservoir with hot water, adding an effervescent composition to the hot water, the effervescent composition causing to be released an amount of gas such that a positive vapor pressure is created in the reservoir, connecting a vapor-concentrating lid to the reservoir, and breathing an amount of humidified air.

The present invention also includes a method for decongesting nasal passages comprising filling a reservoir with hot water, adding an effervescent composition to the hot water, the effervescent composition causing to be released an amount of gas such that a positive vapor pressure is created in the reservoir, connecting a vapor-concentrating lid to the reservoir, and breathing an amount of humidified air.

### BRIEF DESCRIPTION OF THE DRAWINGS

FIG. 1 is a perspective view of the portable vapor inhaler of the present invention.

FIG. 2 is a perspective view of the vapor-concentrating lid of the vapor inhaler of FIG. 1.

FIG. 3 is a perspective view of another embodiment of the vapor-concentrating lid of FIG. 2.

### DETAILED DESCRIPTION

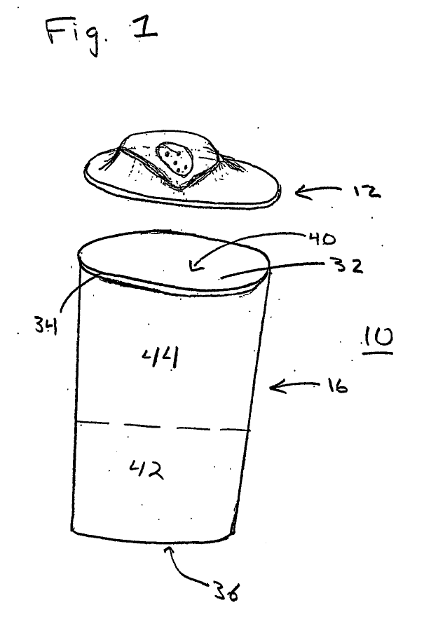

The present invention will be herein described with reference to FIGS. 1-3. As illustrated in FIG. 1, the present invention is a vapor inhaler 10, including a vapor-concentrating lid 12, an effervescent composition 14, and a reservoir 16. The vapor-concentrating lid 12 is removably engagable to the top of the reservoir 16.

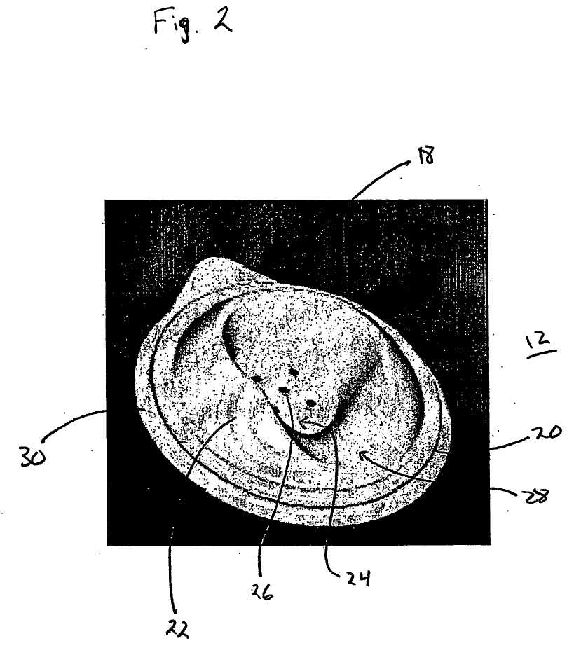

As illustrated in FIG. 2, the vapor-concentrating lid 12 includes a central cover portion 18, a rim engaging portion 20, a shaped wall 22, and a central depressed area 24. The central depressed area 24 further includes one or more vents 26 therein. The shaped wall 22 may further include a shaped depression 28. The wall 22 may be several shapes, such as frusto-conical or dome shaped. The combination of the central depressed area 24 and the shaped depression 28 preferably form an ergonomic shape which allows the user to place his or her nose in close contact with the one or more vents 26, but also protects a majority of the user's face, including his or her eyes, mouth, and skin, from direct exposure to the vapor as it escapes from the one or more vents 26 (as further described below). The vapor-concentrating lid 12 is of a size such that the combination of the central depressed area 24 and the shaped depression 28 conforms approximately to the size and shape of the user's facial structure around the nose, hereinafter referred to as the nasal area Various shaped vapor-condensing lids 12 are shown in FIGS. 2-5.

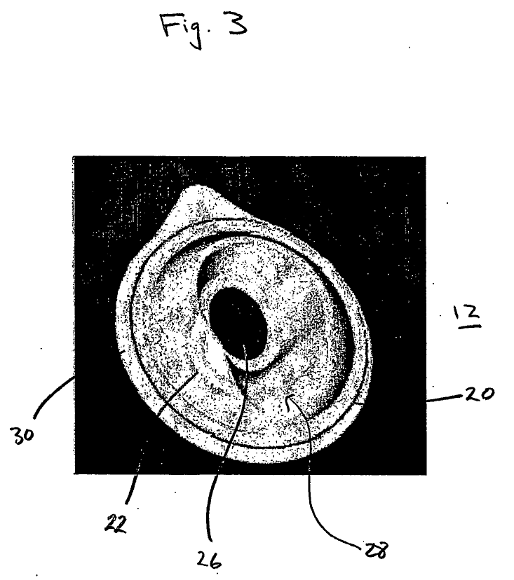

The vapor-concentrating lid 12 is designed to concentrate the emitted air and vapor at the vents 26, and to emit the humidified air and vapor through the vents 26 in the vapor-concentrating lid 12. The vapor-concentrating lid 12 should be of lightweight construction but durable enough to remain intact after attaching to the reservoir. The vapor-concentrating lid 12 may be made from a variety of materials, including, but not limited to, thermoplastics such as polystyrene, polypropylene, and polyethylene terephthalate (PET), or from a variety of synthetic or natural rubbers. Various vapor condensing lids 12 illustrated in FIGS. 2-3 were produced from thin polystyrene plastic using typical vacuum thermoforming techniques. The vapor-condensing lid 12 weighs about 5 grams.

The vapor-concentrating lid 12 should fit tightly onto the reservoir 16, either by close friction fit or threaded screw attachment, so as to form a substantially fluid impermeable barrier. In one embodiment, the rim engaging portion 20 of the vapor-concentrating lid 12 is substantially round such that it can be fitted over a pre-determined and standard sized hot beverage cup. Alternatively, the vapor-concentrating lid 12 may be sized such that it fits over a variety of common cup sizes, such as standard styrofoam or paper cups. In such an embodiment it is not necessary for a reservoir 16 to be provided to the user. In alternative embodiments, the reservoir 16 can be provided along with the vapor-condensing lid 12. In such alternative embodiments the vapor-concentrating lid 12 is sized and shaped to match the opening of the provided reservoir 16.

The vents 26 may be one or more relatively small, substantially centrally located openings, with the desired effect of concentrating the decongesting vapor into an area smaller than the opening of the reservoir. In further embodiments, and as illustrated in FIG. 3, the vents 26 may be one or more larger openings of a variety of shapes. As long as the vents 26 allow the user to breathe or inhale some amount of the humid air from the vapor inhaler 10, the vents 26 are effective for the desired purpose.

As previously described, the reservoir 16 may be a standard sized beverage cup designed to contain hot beverages, such as a styrofoam cup, a plastic cup, a cardboard cup, a paper cup, or a ceramic coffee cup. Such a cup, as illustrated in FIG. 1, normally includes a generally circular opening 32 that includes a lip 34 around the circumference. The cup may also include a bottom 36 and a sidewall 38 whereby an interior reservoir 40 is formed. As may be appreciated, the interior reservoir 40 includes, after the hot water is placed therein, a water repository 42 and an air repository 44. The air repository 44 is where an amount of humidified vapor collects in the reservoir 16 before being inhaled.

Where the reservoir 16 is provided, the reservoir 16 should be relatively small in size so that it can be easily carried, held, stored and filled with water. The reservoir 16 should also be of minimal weight to achieve its function. It should hold a reasonable amount of water, and ideally, when filled with hot water, it should provide sufficient insulation to keep the water hot during the period of use.

Some typical specifications of the reservoir 16 are as follows.

TABLE 1

| | General Requirements | | Preferred Requirements | |
|---|---|---|---|---|
| | Minimum | Maximum | Minimum | Maximum |
| Volume | 30 mL | 2 L | 100 mL | 1 L |
| Height | 5 cm | 30 cm | 8 cm | 15 cm |
| Width | 2 cm | 20 cm | 5 cm | 10 cm |
| Weight | None | 500 g | None | 50 g |

| | | |
|---|---|---|
| Material | Insulating plastic, | Same |
| | fiber or foam | |
| Shape | Any | Traditional drinking cup |

The effervescent composition 14 provides the positive pressure to the humidified air by bubbling released effervescent gases through the hot water in the reservoir 16. Effervescent compositions are commonly used in products as diverse as antacids and denture cleaners and any such system may be utilized effectively in the present invention. Ideally, the effervescent composition 14 would also contain decongesting fragrance materials such as camphor and menthol. The fragrance is preferably perceptible by olfaction for at least about 10 minutes after being placed in water having a temperature of at least about 100° F. A general example of possible specifications for the effervescent composition 14 is given below.

TABLE 2

| | General Requirements | | | | Preferred Requirements | | | |
|---|---|---|---|---|---|---|---|---|
| | Minimum | | Maximum | | Minimum | | Maximum | |
| Weight | 0.1 | g | 100 | g | 1 | g | 5 | g |
| Gas Generated | 1 | mL | 10 | L | 100 | mL | 1 | L |
| Dissolution Time | 1 | sec | 10 | min | 1 | min | 2 | min |

One effervescent composition 14 was made from the following formula using common mixing equipment and techniques.

TABLE 3

| | |
|---|---|
| Ingredient | Wt % |

| | | |
|---|---|---|
| Sorbitol | 20-30% | w/w |
| Citric acid | 20-30% | w/w |
| Sodium bicarbonate | 30-40% | w/w |
| and bicarbonate | | |
| Polyethylene glycol | 3% | w/w |
| Eucalyptus oil | 2.3% | w/w |
| Sodium benzoate | 2% | w/w |
| Menthol | 1.7% | w/w |
| Magnesium oxide | 1% | w/w |
| FD&C Yellow #5 Aluminum | 0.04% | w/w |
| Lake (aka Yellow 5 Lake) | | |
| FD&C Blue #1 Aluminum | 0.02% | w/w |
| Lake (aka Blue 1 Lake) | | |

An effervescent composition 14 according to Table 3 was blended and then made into tablets of about 3.30 g to about 3.65 g each. The tableting is preferably done at lower humidity levels. For example, at 70° F., the relative humidity is preferably below 20% and more preferably is below 15%. The tablets formed had a green and white mottled/speckled appearance, a diameter of approximately 0.833 inches, and a thickness less than about 0.290 inches. The tablets are pressed using conventional tableting techniques, and are preferably pressed to an initial hardness of between about 7 to about 10 kiloponds. After 24 hours the tablets will reach a relatively stable hardness of 18 to 25 kiloponds using a Schleuniger 6D hardness tester or equivalent. The tablet of the effervescent composition 14 according to Table 3 takes between about 30 and about 150 seconds to dissolve, averaging about 100 seconds, when placed in 125 mL of water at 100° F. and between about 30 and about 80 seconds, averaging about 50 seconds, to dissolve when placed in 125 mL of water at 140° F. After 48 hours or more after tableting the tablets will have a friability less than about 2% using a Vankel friabilator or equivalent.

A second effervescent composition 14 was made from the following formula using common mixing equipment and techniques.

TABLE 4

| Ingredient | Wt % |
|---|---|
| Citric Acid | 58% |
| Sodium Bicarbonate | 33% |
| Sodium Carbonate | 3% |
| Aminoacetic acid | 2% |
| Flavor | 2% |
| Menthol | 1% |
| Eucalyptus Oil | 0.5% |
| Camphor | 0.3% |
| Excipients | .2 |

This effervescent composition 14 according to Table 4 was adapted from: Pharmaceutical Dosage Forms: Tablets, Volume I. Lieberman H A, Lachman L, Schwartz J B, Eds, Marcel Dekker, Inc, New York, 1989. The effervescent composition 14 was then compacted into tablets weighing approximately three grams each. The tablets were also found to work effectively for the desired purpose.

The examples of the effervescent compositions 14 may also be used in powder or other forms. Other forms would have different characteristics from a tablet that might be advantageous in some circumstances. Such as, for example, adhering the composition to a water soluble backing.

General Use

In use, the reservoir 16 is filled with hot water of a pre-determined temperature. Preferably the water temperature will be approximately 80° F. to 200° F., and more preferably from 90° F. to 110° F. The air repository 44 takes up the space of the reservoir 16 not occupied by the water repository 42. The vapor-concentrating lid 12 is securely fitted to the reservoir 16 by engagement of the rim engaging portion 20 over the lip 34 of the reservoir 16. The vapor-concentrating lid 12 should be secured to the reservoir 16 such that the vapor-condensing lid 12 and the reservoir 16 form a substantially fluid impermeable barrier. The humidified vapor then begins collecting in the air repository 44 under the vapor-condensing lid 12. The effervescent composition 14, in either powder or tablet form, is then placed into the water through vents 26.

As gases bubble through the water, positive pressure begins to build in the air repository 44. The positive pressure forces the humidified vapor out of the one or more vents 26. At this time, the user places his or her facial area in close contact with the vapor-condensing lid 12 such that the depressed area 24 and the shaped depression 28 loosely engage the nasal area of the user's face. The user then breathes the humidified vapor for a desired period of time or until the effervescent composition 14 stops forcing humidified vapor through the vents 26.

As may be appreciated, the steps for utilizing the present invention can be carried out through a variety of different ways. For example, the effervescent composition 14 could be already present in the reservoir 16 and simply activated by the addition of water to the reservoir 16. The composition 14 could be adhered to the bottom of the reservoir 16 or applied as a film to the inner surface of the reservoir 16. Additionally, the composition 14 can be dropped into the reservoir 16 before or after the vapor-condensing lid 12 is fitted onto the reservoir 16 and before or after the water is added thereto.

### EXAMPLE 1

In one specific example of the current invention, a common Dixie® PerfecTouch™ (Georgia-Pacific Consumer Products, Atlanta, Ga.) 12 ounce paper coffee cup is used as the reservoir 16. This cup meets the preferred characteristics described above in that it is about 12 centimeters tall, has a total volume of about 360 milliliters, weighs about 10 grams, has a top opening of about 9 centimeters, is easily filled, held, and carried, and is sufficiently insulated to keep liquids hot for as long as an hour. The reservoir 16 is then filled with 125 milliliters of very hot tap water, with a temperature of about 122° F. A vapor-concentrating lid 12 is placed thereon by gently pressing the vapor-condensing lid 12 against the top of the reservoir 16. The effervescent composition 14 of Table 4 was added and the humidified vapors were inhaled. Various parameters for the invention, before and after use, are listed below.

TABLE 5

| | | |
|---|---|---|
| Empty Vapor Inhaler Weight | 18 | g |
| Water Filled Vapor Inhaler Weight | 143 | g |
| Initial Water Temperature | 50° | C. |
| Water Temperature after 10 minutes | 45° | C. |
| Tablet Dissolution Time | 70 | sec |
| Weight Lost During Dissolution | 0.2 | g |
| Equivalent Amount of CO₂ Evolved | 100 | mL |

### ALTERNATIVE EMBODIMENTS

In one alternative embodiment, the reservoir 16 may be a collapsible membrane that can be fitted to the vapor-concentrating lid 12, filled with hot water and placed inside of a cup or other container that will insulate the hot water and allow the user to hold the inhaler 10. The flexible membrane may have a stiff rim for attachment of the vapor-condensing lid 12, but otherwise be flexible enough to conform to a variety of container shapes and sizes. Such an embodiment would have the advantages of having a small size, being lightweight, being transportable, and being economically constructed.

In still further embodiments, the reservoir 16 and the vapor-concentrating lid 12 may be substantially one piece. In such an embodiment, the one piece reservoir 16 and the vapor-concentrating lid must have a closeable opening sufficient to fill the reservoir 16 with hot water and to also place the effervescent composition 14 therein. Alternatively, the effervescent composition 14 may be placed in the reservoir 16 prior to completely assembling the one-piece vapor inhaler 10.

In still further embodiments, the combination of the vapor-condensing lid 12, reservoir 16, and composition 14 may be a one use system. In such a system, the effervescent composition 14 may be already affixed to an interior of the reservoir 16 such that simply filling the reservoir 16 activates the effervescent action.

As an alternative to effervescent compositions 14, or in addition to them, any composition or ingredient capable of generating an internal positive pressure within the reservoir may be used. For example, utilization of carbonated liquids to create a positive pressure in the reservoir 16 could be incorporated.

In further embodiments, the reservoir could be lined with a non-insulating material such as aluminum foil to protect the insulating material from contact with the fragrance materials and effervescent solution.

The invention of this application is described above both generically and with regard to specific embodiments. A wide variety of alternatives known to those of ordinary skill in the art can be selected within the generic disclosure. The examples provided herein are not to be limited by the examples, but rather, the claims are considered to provide the complete scope of the invention.

What is claimed is:

1. A portable vapor inhaler comprising:
a reservoir;
a vapor-concentrating lid that is removably attachable to the reservoir; and
an effervescent composition.

2. The portable vapor inhaler of claim 1, wherein the reservoir is a cup.

3. The portable vapor inhaler of claim 2, wherein the cup is selected from the group consisting of an insulated cup, a styrofoam cup, a cardboard cup, a plastic cup, a ceramic cup, and a paper cup.

4. The portable vapor inhaler of claim 1, wherein the reservoir is a collapsible membrane to which the vapor-concentrating lid can be attached, whereby the membrane is filled with water and placed into a container of any similar size and shape.

5. The portable vapor inhaler of claim 1, wherein the vapor-concentrating lid further comprises one or more vents.

6. The portable vapor inhaler of claim 1, wherein the vapor-concentrating lid further comprises a central depressed area of a size and shape whereby during use a user's nasal area is loosely engaged by the central depressed area.

7. The portable vapor inhaler of claim 1, wherein the reservoir and the vapor-concentrating lid form substantially one piece and the vapor-concentrating lid further comprises a closeable opening whereby the effervescent composition and water can be added to the reservoir.

8. The portable vapor inhaler of claim 1, wherein the effervescent composition includes one or more components selected from the group consisting of sodium bicarbonate, sodium carbonate, citric acid, sorbitol, polyethylene glycol, sodium benzoate, magnesium oxide, and aminoacetic acid.

9. The portable vapor inhaler of claim 1, wherein the effervescent composition includes one or more components selected from the group consisting of menthol, eucalyptus oil, camphor, a flavor additive, and an excipient.

10. The portable vapor inhaler of claim 9, wherein the effervescent composition includes a coloring agent.

11. A portable vapor inhaler comprising:
a vapor-concentrating lid; and
an effervescent composition.

12. The portable vapor inhaler of claim 11 further comprising a reservoir, wherein the vapor-concentrating lid is removably attachable to the reservoir.

13. A system for the inhalation of humidified vapor comprising:
a reservoir;
a vapor-concentrating lid that is removably attachable to the reservoir; and
an effervescent composition.

14. A method for the inhalation of humidified vapor comprising:
filling a reservoir with hot water;
adding an effervescent composition to the hot water, the effervescent composition causing to be released an amount of gas such that a positive vapor pressure is created in the reservoir;
connecting a vapor-concentrating lid to the reservoir whereby an amount of humidified air forms in the reservoir; and
inhaling an amount of humidified air emitted through the vapor-concentrating lid.

15. A method of treating cold symptoms comprising:
filling a reservoir with hot water;
adding an effervescent composition to the hot water, the effervescent composition causing to be released an amount of gas such that a positive vapor pressure is created in the reservoir;
connecting a vapor-concentrating lid to the reservoir whereby an amount of humidified air forms in the reservoir; and
treating the cold systems by breathing an amount of humidified air emitted through the vapor-concentrating lid.

16. A method of treating allergy symptoms comprising:
filling a reservoir with hot water;
adding an effervescent composition to the hot water, the effervescent composition causing to be released an amount of gas such that a positive vapor pressure is created in the reservoir;
connecting a vapor-concentrating lid to the reservoir whereby an amount of humidified air forms in the reservoir; and
treating the allergy symptoms by breathing an amount of humidified air emitted through the vapor-concentrating lid.

17. A method of decongesting nasal passages comprising:
filling a reservoir with hot water;
adding an effervescent composition to the hot water, the effervescent composition causing to be released an amount of gas such that a positive vapor pressure is created in the reservoir;
connecting a vapor-concentrating lid to the reservoir whereby an amount of humidified air forms in the reservoir; and
decongesting nasal passages by breathing an amount of humidified air emitted through the vapor-concentrating lid.
